(19) United States
(12) Patent Application Publication
(10) Pub. No.: US 2010/0285534 A1
(43) Pub. Date: Nov. 11, 2010

(54) COMBINED THERMOCHEMICAL PRETREATMENT AND REFINING OF LIGNOCELLULOSIC BIOMASS

(75) Inventors: Colin R. South, Lexington, MA (US); Herve I. Garant, Belmont, MA (US); Richard Lance Martin, San Francisco, CA (US)

Correspondence Address: FOLEY HOAG, LLP;PATENT GROUP, WORLD TRADE CENTER WEST, 155 SEAPORT BLVD, BOSTON, MA, 02110, US

(73) Assignee: MASCOMA CORPORATION, Lebanon, NH (US)

(21) Appl. No.: 12/596,114

(22) PCT Filed: Apr. 18, 2008
(86) PCT No.: PCT/US2008/060835

§ 371 Date: Jul. 27, 2010

Related U.S. Application Data
(60) Provisional application No. 60/925,257, filed on Apr. 19, 2007.

(51) Int. Cl. C12P 7/06; D21C 1/02; C12P 19/00

(52) U.S. Cl. 435/72; 162/68; 435/161

(57) ABSTRACT

One aspect of the present invention relates to a method of processing lignocellulosic material, comprising initial steam pretreatment to give pretreated lignocellulosic material with an average particle size, followed by refining to give refined lignocellulosic material with an average particle size, wherein the average particle of the pretreated lignocellulosic material is greater than the average particle size of the refined lignocellulosic material. In certain embodiments, the lignocellulosic material is selected from the group consisting of grass, switch grass, cord grass, rye grass, reed canary grass, miscanthus, sugar-processing residues, sugar cane bagasse, agricultural wastes, rice straw, rice hulls, barley straw, corn cobs, cereal straw, wheat straw, canola straw, oat straw, oat hulls, corn fiber, stover, soybean stover, corn stover, forestry wastes, recycled wood pulp fiber, sawdust, hardwood, and softwood.

### RELATED APPLICATIONS

This application claims the benefit of priority to U.S. Provisional Patent Application Ser. No. 60/925,257, filed Apr. 19, 2007.

### BACKGROUND OF THE INVENTION

The production of ethanol from lignocellulosic material involves the breakdown and hydrolysis of lignocellulose-containing materials, such as wood, into disaccharides, such as cellobiose, and ultimately monosaccharides, such as glucose and xylose. Microbial agents, including yeasts, then convert the monosaccharides into ethanol in a fermentation reaction which can occur over several days or weeks. Thermal, chemical and/or mechanical pretreatment of the lignocellulose-containing materials can shorten the required hydrolysis and fermentation time and improve the yield of ethanol. Since the first alkaline pre-treatment based on impregnation with sodium hydroxide in the early 1900s, which improved the digestibility of straw, many pre-treatment methods or techniques have been developed for lignocellulosic materials.

A fundamental objective of pre-treatment is to reduce the crystallinity of the cellulose and to dissociate the hemicellulose-cellulose-lignin complex. The digestibility of the cellulose typically increases with the degree of severity of the pre-treatment. This increase in digestibility is often directly related to the increase in the available surface area (ASA) of the cellulose materials, which facilitates the eventual enzymatic attack by enzymes such as cellulases.

Thermochemical pre-treatment processes are among the most effective for improving the accessibility of these materials. An example of such a thermochemical process is described in Spanish patent ES87/6829, which uses steam at a temperature of 200-250° C. in a hermetically sealed reactor to treat previously ground lignocellulosic material. In this process, the reactor is cooled gradually to ambient temperature once the lignocellulosic material is treated. Thermochemical treatment that includes a sudden depressurization of the reactor, called steam explosion treatment, is one of the most effective pre-treatment techniques when it comes to facilitating the eventual action of cellulolytic enzymes. In some instances, the pretreatment protocol incorporates varying concentrations of a catalytic agent (e.g. acid); however, the use of pretreatment technologies characterized by high concentrations of acids is costly due to the need to recover and recycle acid.

It is therefore an object of this invention to provide an improved and yield-efficient way of shortening the required fermentation time and/or improving the yield of ethanol from lignocellulosic biomass. Other objects of the invention will be apparent from the following disclosure, claims, and drawings.

### SUMMARY OF THE INVENTION

Disclosed herein is a method of processing lignocellulosic biomass using a refiner combined with mild pretreatment conditions, which provides high ethanol yields while minimizing or eliminating the need to recover and recycle acid or other added catalysts, and simultaneously reduces the amount of unwanted by-products. Use of a refiner is believed to improve ethanol yield and/or rate by breaking the pretreated cellulose material into smaller particles, which increases susceptibility to enzymatic hydrolysis, thereby increasing the effectiveness of enzymatic hydrolysis and ultimately resulting in greater yield of ethanol and/or increased reactions rates.

One aspect of the invention relates to methods of processing lignocellulosic material through a refiner to improve the yield of ethanol from lignocellulosic material. In certain embodiments, lignocellulosic material may be placed into one or more pre-treatment reactors, then steam may be injected into said one or more pre-treatment reactors, at a temperature, steam pressure, and for a time sufficient to allow the incorporation of the steam into the lignocellulosic material, thereby producing pretreated lignocellulosic material. The pretreated material may be fed through a refiner, wherein the refiner grinds said pretreated material into smaller pieces. Smaller pieces of the refined lignocellulosic material may be more susceptible to enzymatic hydrolysis, resulting in greater yield and/or rate of formation of monomeric sugars and thence ethanol from fermentation.

### BRIEF DESCRIPTION OF THE FIGURES

FIG. 1 depicts as a function of time the yields of monomeric sugars using (1) a continuous pretreatment followed by refinement; and (2) a batch pretreatment without refinement.

FIG. 2 depicts results from simultaneous saccharification and fermentation performed using pretreated and refined hardwood chips in a glucose and xylose fermenting co-culture with excess enzyme.

FIG. 3 depicts a table showing CAFI 2 standard poplar's components and compositions (wt %).

FIG. 4 depicts a table listing key features of CAFI pretreatments.

FIG. 5 depicts a table showing publication of results from CAFI 1.

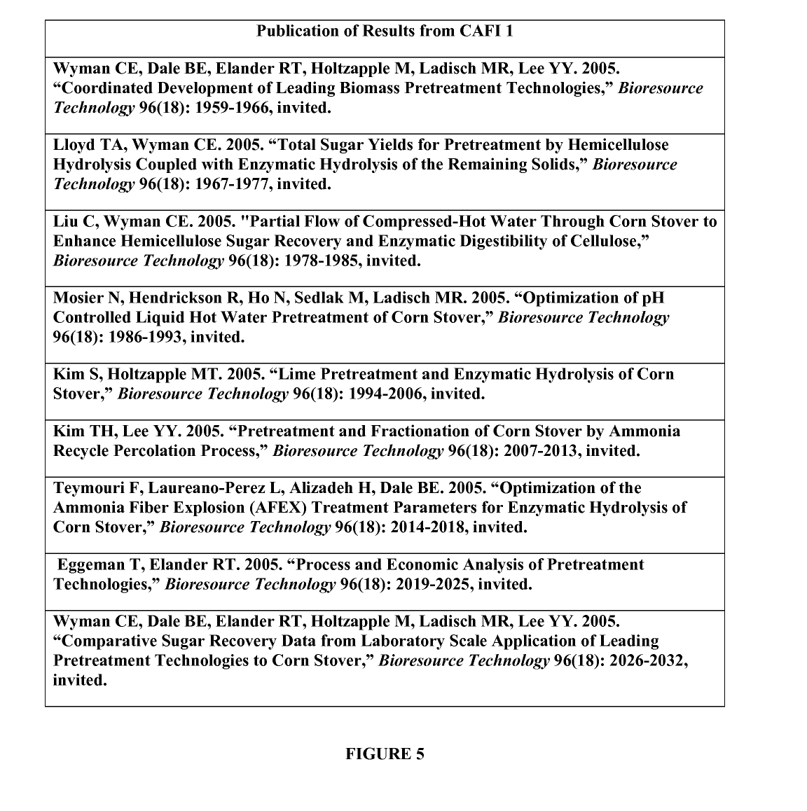

FIG. 6 shows a perspective view of a PeriFeeder™ mechanical steam separator from Metso Paper.

FIG. 7 shows a perspective view of a mechanical steam separator from Andritz.

### DETAILED DESCRIPTION OF THE INVENTION

One aspect of the present invention relates to a process by which steam pretreated lignocellulosic material is processed through a refiner to increase the yield of ethanol in fermentation. Lignocellulosic material may be subjected to steam hydrolysis and fed through a refiner to reduce the particle size of the pretreated material.

The terms “lignocellulosic material” and “lignocellulosic substrate” mean any type of lignocellulosic material comprising cellulose, such as but not limited to non-woody-plant lignocellulosic material, agricultural wastes, forestry residues, paper-production sludge, waste-water-treatment sludge, corn fiber from wet and dry mill corn ethanol plants, and sugar-processing residues.

In a non-limiting example, the lignocellulosic material can include, but is not limited to, grasses, such as switch grass, cord grass, rye grass, reed canary grass, miscanthus, or a combination thereof; sugar-processing residues, such as but not limited to sugar cane bagasse; agricultural wastes, such as but not limited to rice straw, rice hulls, barley straw, corn cobs, wheat straw, canola straw, oat straw, oat hulls, and corn fiber; stover, such as but not limited to soybean stover, corn stover; and forestry wastes, such as but not limited to recycled wood pulp fiber, sawdust, hardwood, softwood, or any combination thereof.

Lignocellulosic materials are composed of mainly cellulose, hemicellulose, and lignin. Generally, a lignocellulosic material, on a dry basis, may contain about 50% (w/w) cellulose, about 30% (w/w) hemicellulose, and about 20% (w/w) lignin. The lignocellulosic material can be of lower cellulose content, for example, at least about 20% (w/w), 30% (w/w), 35% (w/w), or 40% (w/w).

Purified cellulose is a linear, crystalline polymer of beta-D-glucose units. The structure is rigid and harsh treatment is usually required to break down cellulose. Hemicellulose has usually as a main component linear and branched heteropolymers of L-arabinose, D-galactose, D-glucose, D-mannose, D-xylose and L-rhamnose. The composition of hemicellulose varies with the origin of the lignocellulosic material. The structure is not totally crystalline and is therefore usually easier to hydrolyze than cellulose. Examples of lignocellulosic materials considered for ethanol production are hardwood, softwood, forestry residues, agricultural residues, and municipal solid waste (MSW). Examples of hardwoods considered for ethanol production may include, but are not limited to, willow, maple, oak, walnut, eucalyptus, elm, birch, buckeye, beech, and ash. Examples of softwoods considered for ethanol production may include, but are not limited to, southern yellow pine, fir, cedar, cypress, hemlock, larch, pine, and spruce.

Both cellulose and hemicellulose can be used for ethanol production. The pentose content in the raw material is of importance because pentoses are often difficult to ferment to ethanol. To achieve maximum ethanol yield, all monosaccharides should be fermented. Softwood hemicellulose contains a high proportion of mannose and more galactose and glucose than hardwood hemicellulose, whereas hardwood hemicellulose usually contains a higher proportion of pentoses like D-xylose and L-arabinose.

The term “reactor” may mean any vessel suitable for practicing a method of the present invention. The dimensions of the pretreatment reactor may be sufficient to accommodate the lignocellulose material conveyed into and out of the reactor, as well as additional headspace around the material. In a non-limiting example, the headspace may extend about one foot around the space occupied by the materials. Furthermore, the pretreatment reactor may be constructed of a material capable of withstanding the pretreatment conditions. Specifically, the construction of the reactor should be such that the pH, temperature and pressure do not affect the integrity of the vessel.

The size range of the substrate material varies widely and depends upon the type of substrate material used as well as the requirements and needs of a given process. In a preferred embodiment of the invention, the lignocellulosic raw material may be prepared in such a way as to permit ease of handling in conveyors, hoppers and the like. In the case of wood, the chips obtained from commercial chippers may be suitable; in the case of straw it may be desirable to chop the stalks into uniform pieces about 1 to about 3 inches in length. Depending on the intended degree of pretreatment, the size of the substrate particles prior to pretreatment may range from less than a millimeter to inches in length. The particles need only be of a size that is reactive.

### Pretreatment

In certain embodiments, a pretreatment may include a steam hydrolysis where lignocellulosic material is subjected to steam pressure of between 100 psig and 700 psig. A vacuum may be pulled within the reactor to remove air, for example, at a pressure of about 50 to about 300 mbar. Steam may be added to the reactor containing the lignocellulosic material at a saturated steam pressure of between about 100 psig and about 700 psig, or any amount therebetween; for example, the saturated steam pressure may be about 100, 150, 200, 250, 300, 350, 400, 450, 500, 550, 600, 650, or 700 psig. More preferably, a saturated steam pressure from about 140 psig to about 300 psig may be used. When no other chemical is added to the steam during the pretreatment process, unwanted by-products and/or waste material produced in some of the conventional methods are eliminated.

Nevertheless, in certain embodiments, it may be desirable to add a catalyst during or before the pretreatment process. If an acid catalyst is used in a method of the present invention it may be any suitable acid known in the art; for example, but without wishing to be limited in any manner, the acid may be sulfuric acid, sulfurous acid, and/or sulfur dioxide, or a combination thereof. The amount of acid added may be any amount sufficient to provide a pre-treatment of the lignocellulosic material at the chosen pre-treatment temperature. For example, the acid loading may be about 0% to about 12% by weight of the materials, or any amount therebetween; for example, the acid may be loaded at about 0, 1, 2, 3, 4, 5, 6, 7, 8, 9, 10, 11, or 12% by weight of the lignocellulosic materials. In a non-limiting example, the acid is sulfur dioxide, and it is added to the lignocellulosic material by injecting the acid as a vapor to a concentration of about 0.5% to about 4.0% the weight of lignocellulosic material.

The acid and steam may be added in any order that is suitable to the present invention. For example, the acid may be added prior to, simultaneously with, or after the addition or injection of steam into the pre-treatment reactor.

The reactor is maintained at a temperature and pH for a length of time sufficient to make the lignocellulosic material amenable to hydrolysis. The combination of time, temperature, and pH may be any suitable conditions known in the art. In a non-limiting example, the temperature, time and pH may be as described in U.S. Pat. No. 4,461,648, which is hereby incorporated by reference.

The temperature may be about 165° C. to about 220° C., or any temperature therebetween. More specifically, the temperature may be about 175° C. to about 210° C., or about 180° C. to about 200° C., or any temperature therebetween. For example, the temperature may be about 165, 175, 185, 195, 205, 215, or 220° C. Those skilled in the art will recognize that the temperature may vary within this range during the pretreatment. The temperatures refer to the approximate temperature of the process material reactor, recognizing that at a particular location the temperature may be higher or lower than the average temperature.

In some embodiments, the pretreatment temperature may be greater than the glass transition point for lignin. When lignocellulosic material is exposed to a temperature beyond the glass transition point, lignin enters the plastic phase and when cooled, the lignin may adhere to itself in a shape of a ball instead of being wrapped in the cellulose. The result may be that more cellulose is exposed for enzymatic hydrolysis.

The heterogeneous enzymatic degradation of lignocellulosic material is primarily governed by its structural features because (1) cellulose possesses a highly resistant crystalline structure, (2) the lignin surrounding the cellulose forms a physical barrier and (3) the sites available for enzymatic attack are limited. An ideal pretreatment, therefore, would reduce lignin content, with a concomitant reduction in crystallinity and increase in surface area.

The pretreatment time may be in the range of about 5 seconds to about 15 minutes, or any amount of time therebetween; for example, the pretreatment time may be about 5 seconds, 30 seconds, 1, 2, 3, 4, 5, 6, 7, 8, 9, 10, 11, 12, 13, 14, or 15 minutes. The pretreatment time may be less than 5 minutes. The pretreatment time refers to the length of time the material is at an elevated temperature, in some embodiments, between 165° C. and 220° C.

A mild pretreatment such as steam hydrolysis which does not add acid or other catalysts during the pretreatment process may be more economical compared to pretreatments with added catalysts. If no acid is used, the high cost and time associated with recovering and recycling acid is eliminated. Traditionally, acid is recovered by subjecting the acid/steam mixtures to condensation followed by purification of the acid via distillation. A pretreatment process having an acid/steam mixture increases a number of steps, time, and expenses compared to a non-catalytic steam pretreatment. In addition, a non-catalytic steam hydrolysis may produce lignocellulosic material that is more susceptible to enzymatic hydrolysis.

The processes of the present invention can be conducted in continuous, semi-continuous or batch fashion and may involve a solid recycle, liquid recycle and/or steam recycle operation as desired. The processes of this invention are preferably conducted in a continuous fashion.

The processes can be conducted in a single pretreatment zone or in a plurality of pretreatment zones, in series or in parallel; or they may be conducted batchwise or continuously in an elongated tubular zone or series of such zones. The materials of construction and the design of the equipment should be able to withstand said temperatures and pressures. Means to introduce and/or adjust the quantity of biomass or steam introduced batchwise or continuously into the pretreatment zone during the course of the process can be conveniently utilized in the processes especially to maintain the desired ratio of the components. The steps may be effected by the incremental addition of one component to the other. Also, the steps can be combined by the joint addition of components.

Once the desired pretreatment reaction time has elapsed, the pretreatment reaction may be terminated by opening the reactor, which releases the steam pressure and rapidly cools the contents. The pre-treated material may then be removed from the reactor by any appropriate means known in the art; for example, the contents may be removed by conveying, exploding, dropping, washing, or slurrying. Alternately, the pretreated material may be maintained at a pressure above atmospheric prior to further processing.

### Refining

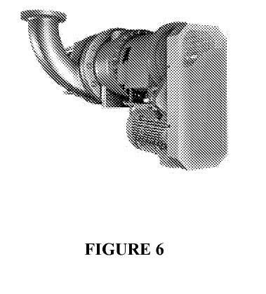

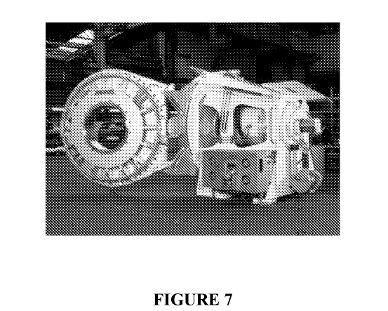

A “refiner” may mean an apparatus capable of reducing a particle in size. One can refine lignocellulosic material as described herein using commercially available refiners. For example, disc refiners made by Metso and Andritz as illustrated in FIGS. 6 and 7 may be appropriate for this purpose. Such apparatus may include single or multiple rotating disks, or be of another design, and may operate either under a set pressure or at atmospheric pressure. A refiner may be a plate grinder, a wood grinder, or a disintegrator. Disintegrators manufactured by Hosokawa may be used to refine pretreated lignocellulosic material.

An embodiment of a feeder-hydrolyzer-refiner system may be able to separate the steam from the fiber before the latter is fed into a refiner. Pulp or hardwood chips and steam may be blown into the inlet of such a device where the steam and the pulp or hardwood chips are separated. Steam may be channeled to a steam outlet and pulp/hardwood chips may be fed through a refiner. A machine may have an inlet, steam outlet, a refiner, and a feeder screw. The feeder screw may aid pulp/hardwood chip and steam separation.

In certain embodiments, the lignocellulosic material may be subjected to steam hydrolysis or other mild autohydrolysis in a reactor. The pretreated lignocellulosic material may then be transported to a separate reactor where the pretreated lignocellulosic material is broken into smaller pieces to increase the surface area of the lignocellulosic material.

In certain embodiments, it may be desirable to operate the pretreatment reactor and the refiner at an elevated pressure. In such configuration, lignin may not have the opportunity to cool and coat the fibers. If lignin does not coat the fiber, it is easier to remove the lignin since it is not attached to the fiber. A higher quality fiber may be produced. Also, reactivity between the refined lignocellulose material and the enzyme may be increased due to the increased fiber surface area as a result of lignin not coating the fiber, and/or size reduction of the lignocellulosic material from the refinement process, and/or disruption of lignin deposition on fiber.

In certain embodiments as described in U.S. Pat. No. 4,427,453, hereby incorporated by reference, untreated lignocellulosic material may be fed into the high pressure reaction vessel by means of a pressure seal-forming, continuously working, worm feeder, in which air and excess fluid, contained in the lignocellulosic material are largely removed. The hydrolysis takes place in the vapour phase in a continuous horizontal tube digester, which serves as the reaction vessel. At the outlet of the digester, the size of the pretreated lignocellulosic material may be reduced.

The term “continuous horizontal tube digester” may include, but is not limited to, digesters that are manufactured by Andritz and Metso, and Black-Clawson Co for the production of cellulose. Such digesters are described by W. Herbert in TAPPI, Vol. 45 (1962) No. 7 S 207A-210A and by U. Lowgren in TAPPI Vol. 45 (1962), No. 7, S. 210A-215A. Such digesters are well known to the skilled artisan.

The term “worm feeder” includes devices known commonly as worm pressers, plug screw feeders, or plug feeders. This device consists of a conical, pressure resistant housing, in which a conical worm with a rotation drive is installed. The housing has at the end of its larger diameter, a generally radial charging opening and ends at its smaller diameter with a generally cylindrically shaped, axial, exit sleeve.

Some of the potential benefits of using the refiner in conjunction with a continuous pretreatment device may be increased reactivity due to 1) disruption of lignin deposition on fiber, and/or 2) increased surface area due to mechanical shearing of the refined lignocellulosic material, and/or 3) increased surface area due to reducing the size of the lignocellulosic material prior to the “explosive” decompression of the material. Furthermore, the refiner may provide a cost effective way to convey the lignocellulosic material from the pretreatment device and assist with forming a seal on the outlet of the pressurized digester.

### Saccharification:

Following refining of the pretreated lignocellulosic material, the refined mixture may be hydrolyzed in the presence of a saccharification enzyme to produce monomeric sugars. The saccharification enzyme may be selected from the following classes of enzymes: cellulases, endoglucanases, exoglucanases, cellobiohydrolases, β-glucosidases, xylanases, endoxylanases, exoxylanases, β-xylosidases, arabinoxylanases, mannases, galactases, pectinases, glucuronidases, amylases, α-amylases, β-amylases, glucoamylases, α-glucosidases, isoamylases.

Saccharification enzymes may be produced synthetically, semi-synthetically, or biologically including using recombinant microorganisms.

In certain embodiments, saccharification and fermentation may be performed simultaneously. In such cases, one or more aforementioned saccharification enzymes may be included in the solution containing one or more biocatalysts selected from bacteria, fungi, and/or yeast.

A recombinant organism may also perform saccharification and fermentation simultaneously. For example, the recombinant organism may be selected from the group consisting of Escherichia coli, Zymomonas mobilis, Bacillus stearothermophilus, Saccharomyces cerevisiae, Clostridia thermocellum, Thermoanaerobacterium saccharolyticum, Pichia stipitis, Escherichia, Zymomonas, Saccharomyces, Candida, Pichia, Streptomyces, Bacillus, Lactobacillus, and Clostridium.

SSF may also be performed using co-cultures of yeast and excess saccharification enzymes.

### EXAMPLE

Enzymatic hydrolysis was run with excess enzyme in order to determine theoretical maximum yield of monomeric sugars using the lignocellulosic material that has been pretreated using steam hydrolysis and passed through a refiner. The percentages reported in Table 1 combine released glucose and xylose.

Hardwood chips were subjected to steam hydrolysis at 160 psig between the resident time of 5 to 10 minutes in a 2 odtpd mechanical pulping system from Andritz. The pretreated lignocellulosic material was reduced in size at the outlet of the system at an elevated temperature of about 188° C. at 160 psig. The refined lignocellulosic materials were then released and depressurized into a separate collection vessel. Subsequently the materials were subjected to enzymatic hydrolysis using cellulase and xylanase enzymes. The maximum theoretical sugar yield of the trial (Method 1) is compared to various pretreatment methods as listed in Table 1.

TABLE 1
Comparison of maximum theoretical sugar yield at saturated
enzyme rate at 24 hr, 48 hr, and 72 hr simultaneous
saccharification and fermentation

| | | 24 Hour | 48 Hour | 72 Hour |
|---|---|---|---|---|
| Method | Pretreatment | Yield | Yield | Yield |
| 1 | Autohydrolysis (Continuous with | 90% | 92% | 95% |
| | Refining) | | | |
| 2 | Autohydrolysis (Batch w/o | 63% | 68% | 70% |
| | Refining) | | | |
| 3 | Autohydrolysis (Published by | — | 80-85% | — |
| | Chornet, continuous w/o refining) | | | |
| 4 | Dilute Acid Hydrolysis (CAFI 2, | 76% | 92% | 95% |
| | poplar) | | | |
| 5 | AFEX (CAFI 2, poplar) | — | — | 60% |

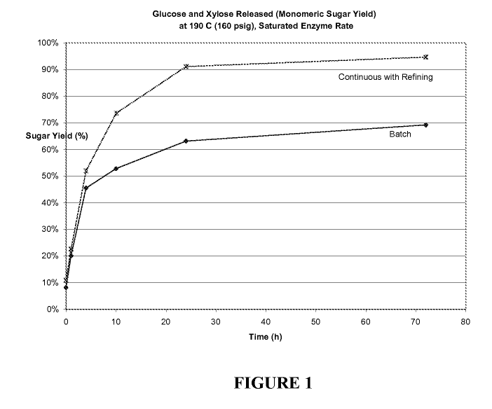

These results indicate that pretreated lignocellulose using continuous steam hydrolysis followed by the refinement process (Method 1) yields more glucose and xylose compared to the batch pretreatment without the refinement process (Method 2). The data shows marked improvement in theoretical yield of sugar from the lignocellulosic material that is pretreated continuously and subjected to the refining process. FIG. 1 graphically illustrates the sugar yield obtained using Method 1 and Method 2.

Results obtained using Method 3 also has a lower maximum theoretical yield, about 80% to about 85%, compared to the pretreated and refined lignocellulosic material having the yield rate of about 92% after 48 hours of SSF (Method 1).

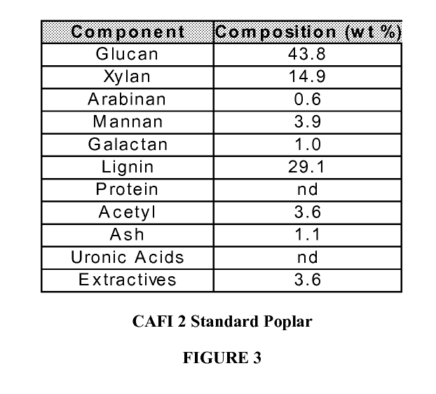

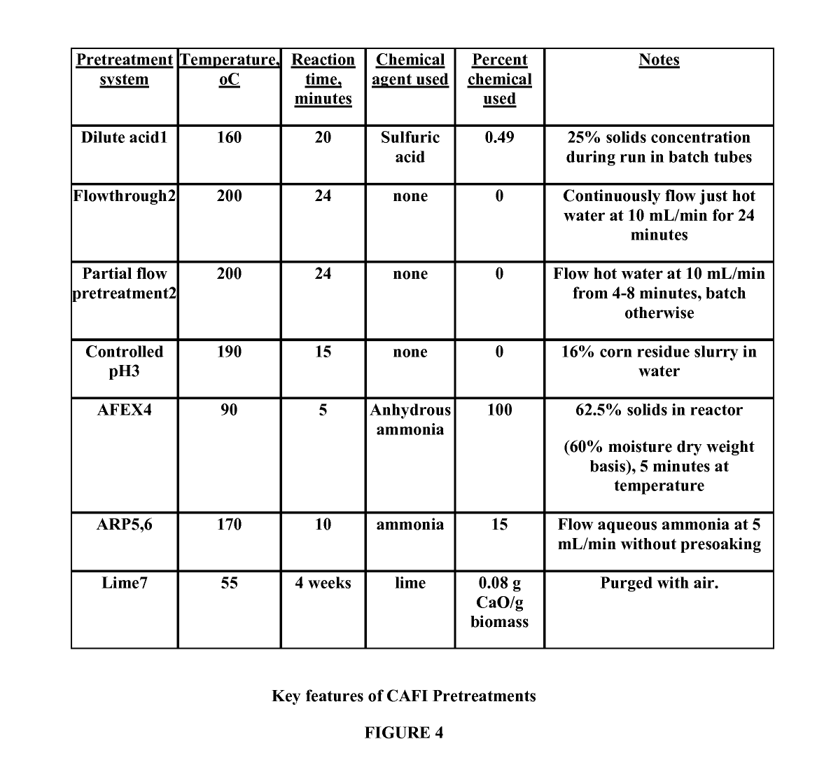

Furthermore, these results indicate that the theoretical yield of monomeric sugars of the pretreated and refined lignocellulosic material (Method 1) compared to the dilute acid hydrolyzed hardwoods after 72 hours of SSF (Method 4) is nearly equivalent, approximately 95% yield of sugar for both cases. Pretreatment details of Method 4 can be found in FIG. 4. FIG. 3 lists CAFI 2 poplar's components and compositions in weight percentage.

CAFI 2 poplar being subjected to AFEX pretreatment resulted in a much lower maximum sugar yield at 72 hours of reaction time, about 60%, compared to results obtained using Method 1 having maximum sugar yield of about 95%.

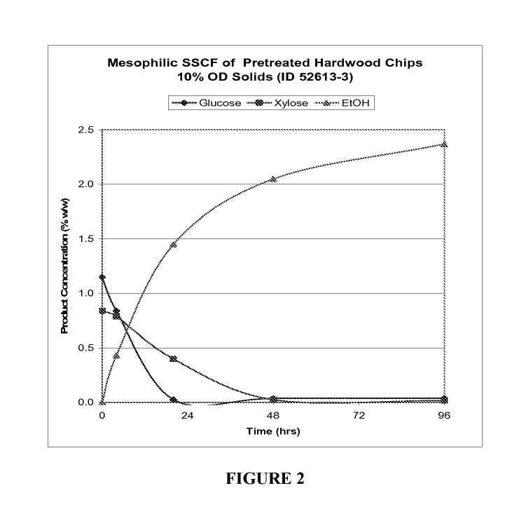

FIG. 2 shows the results of simultaneous saccharification and fermentation trials performed using co-cultures of glucose-fermenting and xylose-fermenting yeasts with excess enzyme. As shown, glucose is converted into ethanol at a faster rate compared to xylose. After 48 hours of fermentation time, it is clear that almost all of glucose and xylose are converted to ethanol.

### Representative Methods of the Invention

According to one embodiment of the present invention, there is provided a method of processing lignocellulosic material through a refiner comprising the steps of: placing lignocellulosic material into one or more pre-treatment reactor, then injecting steam to one or more pre-treatment reactors, at a temperature, steam pressure, and for a time, thereby producing pretreated lignocellulosic material, and subjecting the said pretreated material through a refiner, wherein the refiner grinds the said pretreated material into smaller pieces.

In certain embodiments, the present invention relates to the aforementioned method, wherein said lignocellulosic material contains, on a dry basis, at least about 20% (w/w) cellulose, at least about 10% (w/w) hemicellulose, and at least about 10% (w/w) lignin.

In certain embodiments, the present invention relates to the aforementioned method, wherein said lignocellulosic material is selected from the group consisting of grass, switch grass, cord grass, rye grass, reed canary grass, miscanthus, sugar-processing residues, sugar cane bagasse, agricultural wastes, rice straw, rice hulls, barley straw, corn cobs, cereal straw, wheat straw, canola straw, oat straw, oat hulls, corn fiber, stover, soybean stover, corn stover, forestry wastes, recycled wood pulp fiber, sawdust, hardwood, and softwood, and combinations thereof.

In certain embodiments, the present invention relates to the aforementioned method, wherein said hardwood is selected from the group consisting of willow, maple, oak, walnut, eucalyptus, elm, birch, buckeye, beech, and ash.

In certain embodiments, the present invention relates to the aforementioned method, wherein said softwood is selected from the group consisting of southern yellow pine, fir, cedar, cypress, hemlock, larch, pine, and spruce.

In certain embodiments, the present invention relates to the aforementioned method, wherein said steam pressure is between about 100 psig and about 700 psig.

In certain embodiments, the present invention relates to the aforementioned method, wherein said temperature is between about 165° C. and about 210° C.

In certain embodiments, the present invention relates to the aforementioned method, wherein said temperature is between about 180° C. and about 200° C.

In certain embodiments, the present invention relates to the aforementioned method, wherein said temperature is between about 185° C. and about 195° C.

In certain embodiments, the present invention relates to the aforementioned method, wherein said time is between about 5 seconds and about 15 minutes.

In certain embodiments, the present invention relates to the aforementioned method, wherein said time is less than 5 minutes.

In certain embodiments, the present invention relates to the aforementioned method, further comprising the step or steps of subjecting the refined lignocellulosic material to a saccharification enzyme.

In certain embodiments, the present invention relates to the aforementioned method, wherein said saccharification enzyme is selected from cellulose-hydrolyzing glycosidases consisting of cellulases, endoglucanases, exoglucanases, cellobiohydrolases, β-glucosidases.

In certain embodiments, the present invention relates to the aforementioned method, wherein said saccharification enzyme is selected from hemicellulose-hydrolyzing glycosidases consisting of xylanases, endoxylanases, exoxylanases, β-xylosidases, arabinoxylanases, mannases, galactases, pectinases, glucuronidases.

In certain embodiments, the present invention relates to the aforementioned method, wherein said saccharification enzyme is selected from starch-hydrolyzing glycosidases consisting of amylases, α-amylases, β-amylases, glucoamylases, α-glucosidases, isoamylases.

In certain embodiments, the present invention relates to the aforementioned method, wherein said saccharification system is selected from cellulases, endoglucanases, exoglucanases, cellobiohydrolases, β-glucosidases, xylanases, endoxylanases, exoxylanases, β-xylosidases, arabinoxylanases, mannases, galactases, pectinases, glucuronidases, amylases, α-amylases, β-amylases, glucoamylases, α-glucosidases, isoamylases.

In certain embodiments, the present invention relates to the aforementioned method, further comprising the steps of subjecting said refined lignocellulose material to a saccharification enzyme and biocatalysts which convert sugar to ethanol.

In certain embodiments, the present invention relates to the aforementioned method, wherein said biocatalyst is selected from the group consisting of bacteria, fungi, and yeast.

In certain embodiments, the present invention relates to the aforementioned method, further comprising the steps of subjecting said refined lignocellulosic material to a recombinant organisms having characterizations of saccharification enzyme and a yeast.

In certain embodiments, the present invention relates to the aforementioned method, wherein said recombination organisms is selected from the group consisting of Escherichia coli, Zymomonas mobilis, Bacillus stearothermophilus, Saccharomyces cerevisiae, Clostridia thermocellum, Thermoanaerobacterium saccharolyticum, Pichia stipitis, Escherichia, Zymomonas, Saccharomyces, Candida, Pichia, Streptomyces, Bacillus, Lactobacillus, and Clostridium.

### INCORPORATION BY REFERENCE

All of the U.S. patents and U.S. published patent applications cited herein are hereby incorporated by reference. In addition, U.S. Pat. No. 4,136,207 is hereby incorporated by reference; U.S. Pat. No. 4,427,453 is hereby incorporated by reference; U.S. Pat. No. 4,600,590 is hereby incorporated by reference; U.S. Pat. No. 5,037,663 is hereby incorporated by reference; U.S. Pat. No. 5,171,592 is hereby incorporated by reference; U.S. Pat. No. 5,473,061 is hereby incorporated by reference; U.S. Pat. No. 5,865,898 is hereby incorporated by reference; U.S. Pat. No. 5,939,544 is hereby incorporated by reference; U.S. Pat. No. 6,106,888 is hereby incorporated by reference; U.S. Pat. No. 6,176,176 is hereby incorporated by reference; U.S. Pat. No. 6,348,590 is hereby incorporated by reference; U.S. Pat. No. 6,392,035 is hereby incorporated by reference; U.S. Pat. No. 6,416,621 is hereby incorporated by reference; U.S. Pat. No. 7,109,005 is hereby incorporated by reference; U.S. Pat. No. 7,198,925 is hereby incorporated by reference; U.S. published patent application 2005/0065336 is hereby incorporated by reference; and U.S. published patent application 2006/0024801 is hereby incorporated by reference; and U.S. published patent application 2007/0031953 is hereby incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method of processing lignocellulosic material, comprising the steps of:
(a) placing a sample of lignocellulosic material in a pre-treatment reactor;
(b) injecting steam into said pre-treatment reactor, at a temperature, a steam pressure, and for a time, thereby producing pretreated lignocellulosic material with an average particle size; and
(c) processing said pretreated lignocellulosic material in a refiner to give a refined lignocellulosic material, wherein the average particle of said pretreated lignocellulosic material is greater than the average particle size of said refined lignocellulosic material.

2. The method of claim 1, wherein said lignocellulosic material contains, on a dry basis, at least about 20% (w/w) cellulose, at least about 10% (w/w) hemicellulose, and at least about 10% (w/w) lignin.

3. The method of claim 1, wherein said lignocellulosic material is selected from the group consisting of grass, switch grass, cord grass, rye grass, reed canary grass, miscanthus, sugar-processing residues, sugar cane bagasse, agricultural wastes, rice straw, rice hulls, barley straw, corn cobs, cereal straw, wheat straw, canola straw, oat straw, oat hulls, corn fiber, stover, soybean stover, corn stover, forestry wastes, recycled wood pulp fiber, sawdust, hardwood, and softwood.

4. The method of claim 1, wherein said lignocellulosic material is hardwood; and said hardwood is selected from the group consisting of willow, maple, oak, walnut, eucalyptus, elm, birch, buckeye, beech, and ash.

5. The method of claim 1, wherein said lignocellulosic material is hardwood, and said hardwood is willow.

6. The method of claim 1, wherein said lignocellulosic material is softwood; and said softwood is selected from the group consisting of southern yellow pine, fir, cedar, cypress, hemlock, larch, pine, and spruce.

7. The method of claim 1, wherein said lignocellulosic material is softwood, and said softwood is southern yellow pine.

8. The method of claim 1, wherein said steam pressure is between about 100 psig and about 700 psig.

9. The method of claim 1, wherein said temperature is between about 165° C. and about 210° C.

10-11. (canceled)

12. The method of claim 1, wherein said time is between about 5 seconds and about 15 minutes.

13. (canceled)

14. The method of claim 1, further comprising the step of exposing said refined lignocellulosic material to a saccharification enzyme, thereby producing a saccharified product mixture.

15. The method of claim 14, wherein said saccharification enzyme is selected from the group consisting of cellulases, endoglucanases, exoglucanases, cellobiohydrolases, and β-glucosidases.

16. The method of claim 14, wherein said saccharification enzyme is selected from the group consisting of xylanases, endoxylanases, exoxylanases, β-xylosidases, arabinoxylanases, mannases, galactases, pectinases, and glucuronidases.

17. The method of claim 14, wherein said saccharification enzyme is selected from the group consisting of amylases, α-amylases, β-amylases, glucoamylases, α-glucosidases, and isoamylases.

18. The method of claim 14, wherein said saccharification enzyme is selected from the group consisting of cellulases, endoglucanases, exoglucanases, cellobiohydrolases, β-glucosidases, xylanases, endoxylanases, exoxylanases, β-xylosidases, arabinoxylanases, mannases, galactases, pectinases, glucuronidases, amylases, α-amylases, β-amylases, glucoamylases, α-glucosidases, and isoamylases.

19. The method of claim 14, further comprising the step of subjecting said saccharified product mixture to an organism that produces ethanol as a metabolite; wherein said organism is selected from the group consisting of bacteria, fungi, and yeast.

20. (canceled)

21. The method of claim 19, wherein said organism is a yeast.

22. The method of claim 1, further comprising the step of exposing said refined lignocellulosic material to a recombinant organism that produces ethanol as a metabolite and produces a saccharification enzyme.

23. The method of claim 22, wherein said recombinant organism is selected from the group consisting of Escherichia coli, Zymomonas mobilis, Bacillus stearothermophilus, Saccharomyces cerevisiae, Clostridia thermocellum, Thermoanaerobacterium saccharolyticum, Pichia stipitis, Escherichia, Zymomonas, Saccharomyces, Candida, Pichia, Streptomyces, Bacillus, Lactobacillus, and Clostridium.

24. The method of claim 22, wherein said recombinant organism is a yeast.

25. The method of claim 22, wherein said saccharification enzyme is selected from the group consisting of cellulases, endoglucanases, exoglucanases, cellobiohydrolases, and β-glucosidases.

26. The method of claim 22, wherein said saccharification enzyme is selected from the group consisting of xylanases, endoxylanases, exoxylanases, β-xylosidases, arabinoxylanases, mannases, galactases, pectinases, and glucuronidases.

27. The method of claim 22, wherein said saccharification enzyme is selected from the group consisting of amylases, α-amylases, β-amylases, glucoamylases, α-glucosidases, and isoamylases.

28. The method of claim 22, wherein said saccharification enzyme is selected from the group consisting of cellulases, endoglucanases, exoglucanases, cellobiohydrolases, β-glucosidases, xylanases, endoxylanases, exoxylanases, β-xylosidases, arabinoxylanases, mannases, galactases, pectinases, glucuronidases, amylases, α-amylases, β-amylases, glucoamylases, α-glucosidases, and isoamylases.
